(19) United States
(12) Patent Application Publication
(10) Pub. No.: US 2010/0121161 A1
(43) Pub. Date: May 13, 2010

(54) Ambulatory Urodynamics

(75) Inventor: David W. Robertson, Framingham, MA (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/595,999

(22) PCT Filed: Mar. 5, 2008
(86) PCT No.: PCT/US08/02903

§ 371 Date: Oct. 15, 2009

Related U.S. Application Data
(60) Provisional application No. 60/923,835, filed on Apr. 17, 2007.

(51) Int. Cl. A61B 5/07

(52) U.S. Cl. 600/302

(57) ABSTRACT

An ambulatory urodynamic data gathering system includes one or more sensors disposed in the gastrointestinal tract, a sensor disposed in the bladder, a sensor disposed at the entrance to the urethra, and a data recorder worn by the patient that is disposed around the patient's waist. The sensors transmit data to the recorder unit, which stores the received data for subsequent analysis. The recorder unit also activates the various sensors as necessary to obtain information at certain predetermined intervals to limit battery usage. The recorder unit can also interrogate the various sensors to obtain the information. In one exemplary embodiment, the ambulatory urodynamic data gathering system includes a method and device for simultaneously recording radio transmitted pressure measurements from a swallowed self-contained capsule (pressure monitor, signal processing, transmitter, power source or coupling antenna), and a self-contained unit placed in the bladder, for the purpose of deriving urodynamic information. The system of the present invention enables collection of pressure wave information above the waist of the patient so that a doctor can subsequently analyze the data to determine where the problem may lie.

### FIELD OF THE INVENTION

The present invention relates generally to methods and apparatuses for obtaining measurements from patients, and more particularly to a method and apparatus for obtaining measurements from a patient's internal systems.

### BACKGROUND

When a patient has a problem with urine leakage or blocked urine flow urodynamic testing can be conducted to determine precisely where the problem exists. Several muscles, organs, and nerves interact to collect, store and release urine. The kidneys form urine by filtering wastes and extra water from the bloodstream. Tubes called the ureters carry urine from the kidneys to the bladder. Normally, urine flows in one direction out from the kidneys to the bladder. Infections can occur if urine backs up toward the kidneys.

The bladder—a hollow muscular organ shaped like a balloon—resides in the pelvis and is held in place by ligaments that are attached to other organs and to the pelvic bones. The bladder stores urine until one is ready to empty it. The bladder swells into a round shape when full and gets smaller as the bladder empties. A healthy bladder can hold up to 16 ounces (2 cups) of urine comfortably for 2 to 5 hours.

The bladder opens into the urethra—a tube that allows urine to pass outside the body. Circular muscles called sphincters close tightly to keep urine from leaking. The involuntary leakage of urine is called incontinence.

Nerves in the bladder indicate when it is time to empty your bladder. When the bladder begins to fill with urine, a sensation arises that one needs to urinate. The sensation becomes stronger as the bladder continues to fill and reaches its limit. At that point, nerves in the bladder send a message to the brain, and the urge to urinate intensifies.

When ready to urinate, the brain signals the sphincter muscles to relax. At the same time, the brain signals the bladder muscles to tighten, squeezing urine out. Urine can then leave the bladder through the urethra. When these signals occur in the correct order, normal urination occurs.

Problems in the urinary system can be caused by aging, illness, or injury. The muscles in the ureters, bladder, and urethra tend to become weaker with age. Urinary infections may increase because as the bladder muscles weaken, these muscles cannot empty the bladder completely. Also, weakening of the muscles of the sphincters and the pelvis can cause incontinence, because the sphincter cannot remain tight enough to hold urine in the bladder or does not have enough support from the pelvic muscles.

Urodynamics constitutes the study of how the body stores and releases urine. Urodynamic tests enable a doctor to see how well the bladder and sphincter muscles work and can help explain symptoms such as: incontinence; frequent urination; sudden, strong urges to urinate; problems starting a urine stream; painful urination; problems emptying your bladder completely; and recurrent urinary tract infections.

Urodynamic tests, usually performed in a physician's office, are used to measure the volume and pressure of urine in the bladder and to evaluate the flow of urine. They are particularly useful for the diagnosis of intrinsic sphincter deficiency and uncertain cases of mixed, overflow, urgency, or total incontinence. Additional tests may be conducted if symptoms indicate that blockage is caused by a condition other than BPH.

Urodynamic tests may involve imaging equipment that films urination or may be as simple as urinating behind a curtain while a doctor or nurse listens. However, these tests are not as precise as desired when attempting to identify a source of a problem with urination.

More precise testing typically involves insertion of sensors and data wires while the patient urinates in front of the doctor or nurse. However, these invasive tests are uncomfortable for the patient and often result in misleading data, as the data is not necessarily representative of normal urination activities.

The present invention is therefore directed to the problem of developing a method and apparatus for precisely determining a cause of urinary or gastrointestinal problems that can provide accurate data without requiring invasive or obtrusive testing.

### SUMMARY OF THE INVENTION

The present invention solves these and other problems by providing a method and device for obtaining detailed and direct measurements of the forces acting upon one's gastrointestinal tract and bladder before, during and after specific urinary activities, including incontinence, which device can be worn by a patient during the patient's normal activities, including outside of the medical facility. By recording data from ingested sensors and transmitters in the GI tract along with a volume/pressure sensor disposed in the bladder, in combination with a leak or urine sensor disposed at the bladder, precise measurement information can be correlated with urinary activities to thereby identify a potential source of urological problems. Thus, the method and apparatus herein can be employed while a patient is carrying on with his or her normal daily activities (within some obvious constraints).

Further aspects of the present invention are enumerated below:

Aspect 1. An apparatus for obtaining data from a patient comprising: (a) an ingestible capsule to sense pressure and to transmit pressure measurements from a gastrointestinal tract of the patient; (b) a bladder unit to sense pressure and to transmit bladder volume data from a bladder of the patient; (c) a leak sensor to be disposed at an outlet of the urethra to sense a presence of urine and to transmit an indication of such presence; and (d) a body worn recorder unit to receive pressure measurements from the ingestible capsule, pressure and volume measurements from the bladder unit, and urine presence indications from the leak sensor and to record the measurements.

Aspect 2. The apparatus according to aspect 1, wherein the bladder unit further comprises: (a) an oil-filled reservoir to float the bladder unit against a wall of the bladder; (b) a ultrasonic sensor to transmit and receive an ultrasonic signal; (c) a processor to measure a time needed to receive a reflection of the transmitted ultrasonic signal, thereby measuring a distance to an opposite wall of the bladder; and (d) a transmitter to transmit the time to the recorder unit.

Aspect 3. The apparatus according to aspect 1, wherein the ingestible capsule further comprises: (a) a pressure sensor to measure pressure; and (b) a transmitter to transmit pressure data to the recorder unit.

Aspect 4. The apparatus according to aspect 1, wherein the leak sensor further comprises: (a) a leakage sensor to detect a presence of urine; and (b) a transmit to transmit urine presence signals to the recorder unit.

Aspect 5. The apparatus according to aspect 1, wherein the body worn recorder unit further comprises a memory to store received data from each of the bladder unit, the ingestible capsule, and the leakage sensor.

Aspect 6. The apparatus according to aspect 1, further comprising a flow rate sensor disposed in a toilet of the patient and transmitting flow rate data to the recorder unit during patient urination.

Aspect 7. The apparatus according to aspect 6, wherein the flow rate sensor further comprises a transmitter transmitting flow rate data to the recorder unit.

Aspect 8. The apparatus according to aspect 1, wherein the bladder unit further comprises: (a) an inflatable housing that is remotely inflatable after insertion of the bladder unit into the bladder; (b) a gas source to emit a gas upon remote activation to inflate the inflatable housing; and (c) a degradable suture sewn into the inflatable housing to maintain an airtight seal around the housing, the degradable suture deteriorating after a predetermined time inside the bladder and causing deflation of the inflatable housing upon the deterioration.

Aspect 9. The apparatus according to aspect 1, wherein the gas source for the bladder unit further comprises: (a) a first compartment; (b) a liquid stored in the first compartment; (c) a second compartment; (d) a plurality of gas-releasing granules stored in the second compartment; (e) a heat breakable seal separating the first compartment from the second compartment; (f) an electric current path disposed along the seal; and (g) a remotely activatable power source coupled to the electric current path to cause an electric current to pass through the electric current path disposed along the seal and to heat the seal thereby breaking the seal and merging the liquid with the granules.

Aspect 10. A method for obtaining urodynamic data from a patient comprising: (a) coupling pressure information from an ingestible capsule disposed in a gastrointestinal tract of the patient to a body worn recorder unit disposed on the patient's body; (b) coupling pressure and volume information from a unit disposed in a bladder of the patient to the body worn recorder unit; and (c) coupling leak sense information from a leak sensor disposed at an entrance to the patient's urethra to the body worn recorder unit.

Aspect 11. The method according to aspect 10, further comprising inflating remotely the bladder unit after inserting the bladder unit into the bladder.

Aspect 12. The method according to aspect 10, further comprising storing pressure and volume data received from the ingestible capsule and the bladder unit in the recorder unit.

Aspect 13. A method for obtaining urological data from a patient comprising: (a) inserting a pressure sensor and volume sensor in the bladder; and (b) inflating the pressure and volume sensor after insertion.

Aspect 14. The method according to aspect 13, further comprising providing an ingestible capsule that includes a pressure sensor and transmitter to be ingested by the patient.

Aspect 15. The method according to aspect 13, further comprising placing a leak sensor at an entrance to the urethra.

Aspect 16. The method according to aspect 13, further comprising placing a flow rate sensor in the patient's toilet.

Aspect 17. The method according to aspect 14, further comprising receiving pressure readings from the ingestible capsule.

Aspect 18. The method according to aspect 13, further comprising receiving pressure and volume readings from the bladder sensor.

Aspect 19. The method according to aspect 15, further comprising receiving a leakage indication upon occurrence from the leak sensor.

Aspect 20. The method according to aspect 16, further comprising receiving flow rate data from the flow rate sensor.

Aspect 21. The method according to aspect 13, further comprising: (a) providing an ingestible capsule that includes a pressure sensor and transmitter to be ingested by the patient; (b) placing a leak sensor at an entrance to the urethra; (c) placing a flow rate sensor in the patient's toilet; and (d) controlling the leak sensor, the flow rate sensor, the bladder sensor and the ingestible sensor with an external recorder unit.

Aspect 22. The method according to aspect 13, further comprising deflating the bladder unit upon completion of testing.

Other aspects of the present invention will be apparent upon review of the following drawings in light of the detailed description.

### BRIEF DESCRIPTION OF THE DRAWINGS

FIG. 1 depicts a diagram of an exemplary embodiment of an apparatus for obtaining urodynamic information from a patient according to one aspect of the present invention.

FIG. 2 depicts a flow chart of an exemplary embodiment of a method for obtaining urodynamic data from a patient according to another aspect of the present invention.

FIG. 3 depicts a block diagram of an exemplary embodiment of a bladder unit for use in the apparatus shown in FIG. 1 according to yet another aspect of the present invention.

FIG. 4 depicts a block diagram of an exemplary embodiment of an ingestible capsule for use in the apparatus shown in FIG. 1 according to yet another aspect of the present invention.

FIG. 5 depicts a block diagram of an exemplary embodiment of a recorder unit for use in the apparatus shown in FIG. 1 according to yet another aspect of the present invention.

FIG. 6 depicts a block diagram of an exemplary embodiment of a leakage sensor for use in the apparatus shown in FIG. 1 according to yet another aspect of the present invention.

FIG. 7 depicts a block diagram of an exemplary embodiment of a flow rate sensor for use in the apparatus shown in FIG. 1 according to yet another aspect of the present invention.

### DETAILED DESCRIPTION

It is worthy to note that any reference herein to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

### System Description

According to one aspect of the present invention, an ambulatory urodynamic data gathering system includes one or more sensors disposed in the gastrointestinal tract, a sensor disposed in the bladder, a sensor disposed at the entrance to the urethra, a flow data sensor disposed in a patient's toilet, and a data recorder worn by the patient that is disposed, for example, around the patient's waist.

The three sensors transmit data to the recorder unit, which stores the received data for subsequent analysis. The recorder unit also controls and activates the various sensors as necessary to obtain information at certain predetermined intervals to limit battery usage. The recorder unit can also interrogate the various sensors to obtain the information. As the distance between the various sensors and the recorder unit is relatively short, the transmissions from each of these sensors to the recorder unit can be accomplished using a variety of techniques, including but not limited to RF transmission, acoustic transmission, and communication technology employed in RF identification tags.

In one exemplary embodiment, the ambulatory urodynamic data gathering system includes a method and device for simultaneously recording radio transmitted pressure measurements from a swallowed self-contained capsule (which includes a pressure monitor, signal processing, transmitter, power source or coupling antenna), and a self-contained unit placed in the bladder, for the purpose of deriving urodynamic information. Other potential sensors include a flow rate sensor disposed in the patient's toilet to transmit flow rate data to the recorder unit when the patient is urinating.

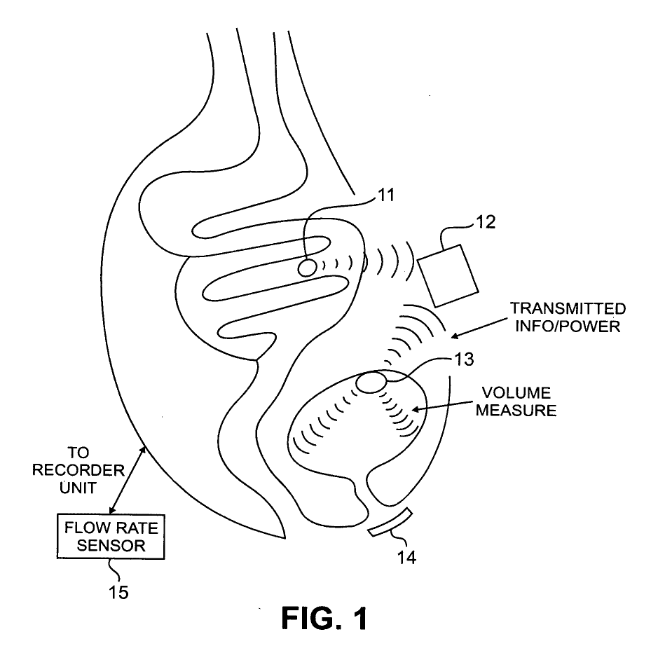

Referring to FIG. 1, shown therein is an exemplary embodiment of an apparatus for obtaining measurement information from a patient according to one aspect of the present invention. The system of the present invention enables collection of pressure wave information above the waist of the patient so that a doctor can subsequently analyze the data to determine where a potential source of urinary problems may exist.

### Ingestible Capsule

An example of an ingestible capsule is disclosed in U.S. Pat. No. 4,844,076, which is hereby incorporated by reference as if repeated herein in its entirety, including the drawings. Another example of an ingestible capsule is disclosed in U.S. Pat. No. 5,279,607, which is also hereby incorporated by reference as if repeated herein in its entirety, including the drawings. Yet another example of an ingestible capsule comprises U.S. Pat. No. 5,604,531, which includes a pressure sensor, which is also hereby incorporated by reference as if repeated herein in its entirety, including the drawings.

Still another example of an ingestible sensor is disclosed in WO 02/095,351 A2, which discloses a floating in vivo sensing device that has a specific gravity of one, which is also hereby incorporated by reference as if repeated herein in its entirety, including the drawings. Such a device can be used to incorporate a pressure sensing mechanism, a very small battery, a transmitter and a receiver along with a limited capability processor to operate the sensor.

An exemplary embodiment of the ingestible capsule employs an ImPressure pressure sensor (Remon Medical Technologies, Caesarea, Israel). The sensor is a miniaturized device that measures 3 mm×9 mm×1.5 mm. It is an ultrasound-based technology that remains quiescent until acoustically activated. Once activated, acoustic energy is converted into electrical energy and a pressure measurement is made. The measurement is transferred to the monitor through acoustic energy.

Multiple ingestible capsules can be swallowed at predetermined intervals as necessary to continuously detect pressure gradients in the gastrointestinal tract. This enables the data recorder to record as detailed a history of the patient's gastrointestinal tract as desired while the patient undergoes his or her daily routine. This enables the doctor to make a better diagnosis as to a source of urinary or other problems.

Shown in FIG. 4 is an exemplary embodiment 40 of an ingestible capsule according to another aspect of the present invention. The ingestible capsule 40 includes a housing 42 which cannot be digested by the body so that the capsule is simply eventually excreted in whole. The capsule 40 includes a pressure sensor 41, a transmitter 44 and a battery 43. The pressure sensor or transmitter may include a simple processor (not shown) to enable activation and response to interrogations by the recorder unit to transmit pressure data, or to do so on some predetermined program.

### Bladder Unit

The bladder unit 13 senses and transmits pressures as well as bladder volume. The bladder volume can be calculated by measuring the distance to the opposite wall of the bladder, which can be accomplished by emitting an ultrasonic signal (or other signal, such as infrared) and determining the time it takes for a reflection to return. Once the distance is determined, known formulae can be employed to calculate the volume of liquid in the bladder. This measurement would be performed frequently enough to derive flow rates of urine during the micturition cycle.

The self-contained bladder unit 13, for example, could be floated in an oil-filled capsule that would float to a point against the bladder wall, giving a reference point from which to measure the distance to the opposite walls (furthest distance).

To insert the bladder unit, the device can be made small to enable simple manual insertion by a urologist. Once inserted, the bladder unit can be activated to inflate itself to prevent the bladder unit from passing out of the bladder. Once the testing is completed, the bladder unit can be deflated to enable the unit to be easily passed out of the bladder through the urethra.

One technique for activating the bladder unit involves inflating the bladder unit externally via a tether, which can be removed and sealed once the unit is inflated.

As another example, the bladder unit can have a small compressed gas source that has a seal that can be opened externally by heat or electric current. Upon unsealing, the compressed gas would expand and inflate the bladder unit.

As yet another example, the bladder unit may incorporate gas-releasing granules, such as crystalline sodium bicarbonate, E-Z GasII effervescent granules by EZEM of New York, United States or similar oxygen releasing granules. These granules release gas (such as carbon dioxide or oxygen) upon contacting liquid. In one exemplary embodiment, the bladder unit includes two compartments—one compartment containing gas-releasing granules (e.g., 100 mg of granules) and the other compartment containing a small amount of aqueous liquid (such as 0.1 cubic centimeters of water or saline solution). The compartments are then maintained separate until the appropriate moment, at which time the compartments are merged and the liquid contacts the gas-releasing granules thereby causing gas to be released and inflate the bladder unit to a size to prevent the unit from passing through the hole in the bladder. Heating of a wax seal can be employed to merge the two compartments, which heat can be generated from an electric current, for example, activated remotely.

According to another aspect of the present invention, once inflated, the bladder unit can remain inflated until degradable sutures (or other degradable members) degrade, thereby freeing the gas and deflating the bladder unit. This would enable the bladder unit to be inserted for a predictable period of time and the automatically be passed outside the bladder once the degradable sutures predictably degraded causing deflation of the bladder unit and enabling the bladder unit to pass through the opening in the bladder.

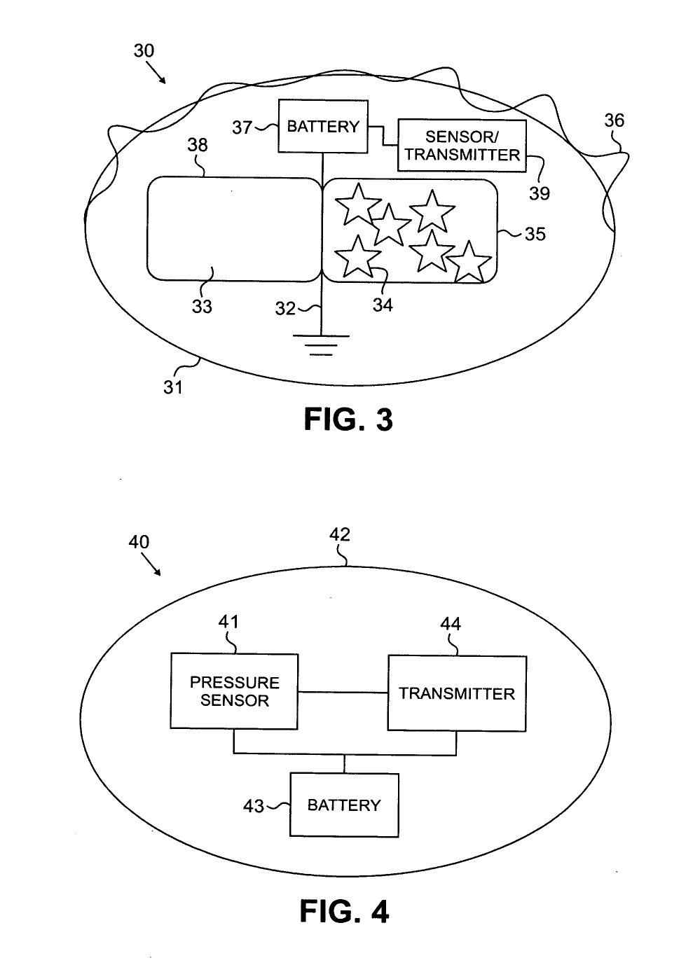

Shown in FIG. 3 is an exemplary embodiment of a bladder unit 30. The bladder unit 30 includes an external housing 31 made of a balloon-like material that expands when inflated. The housing 31 may also include an oil (not shown) to cause the structure 30 to float towards one wall of the bladder. Two compartments 35, 38 store the makings of the gas to inflate the housing 31. A first compartment 35 stores gas-releasing granules 34. A second compartment 38 stores a liquid 33, such as water or the oil mentioned above. A seal 32 between the two compartments maintains the liquid and granules separate. The seal can be composed of a wax and a metal wire. The wire is coupled to a battery 37 and ground. A sensor/transmitter 39 activates the battery causing a current to flow through the seal, thereby melting the wax and opening the seal between the two compartments. Upon the liquid 33 meeting the gas-releasing granules 34, gas is released and the housing 31 is inflated sufficiently to maintain the structure in the bladder. Degradable sutures 36 maintain the seal of the housing 31. After a known time, the sutures 36 deteriorate sufficiently, which in turn opens the housing 31 to the exterior and releases the gas thereby deflating the housing 31, and enabling the structure to pass out of the bladder in the normal manner. The sutures 36 can be simply tied to the end of the “balloon” 31 or can be interwoven amongst the “balloon” 31 to sew two halves of the housing together in a closed seam, much like that of a football. The sensor/transmitter 39 remotely communicates with the recorder unit and receives an instruction to energize the seal to initiate inflation.

### Central Recorder Unit

Signals from both self-contained units 11, 13 would be picked up (and activated) by a central unit 12 worn on the outside of the body that would record and process the signals in a manner to derive the required urodynamic profiles, such as detrusor pressures and flow rates. This “Holter” type recorder 11 can operate for an extended period of time. An example of a Holter type recorder is disclosed in U.S. Pat. No. 6,456,872, which is hereby incorporated by reference as if repeated herein in its entirety, including the drawings. The useful data would be collected as long as the pressure sensor 11 transits the gastrointestinal system, and could continue as a series of self-contained units 11 are swallowed.

Upon receipt of any data, the recorder unit applies a time stamp to the data to enable subsequent correlation of data from each of the various sensors. Time plots can then be generated and placed in a column as is done, for example, for electrocardiogram plots.

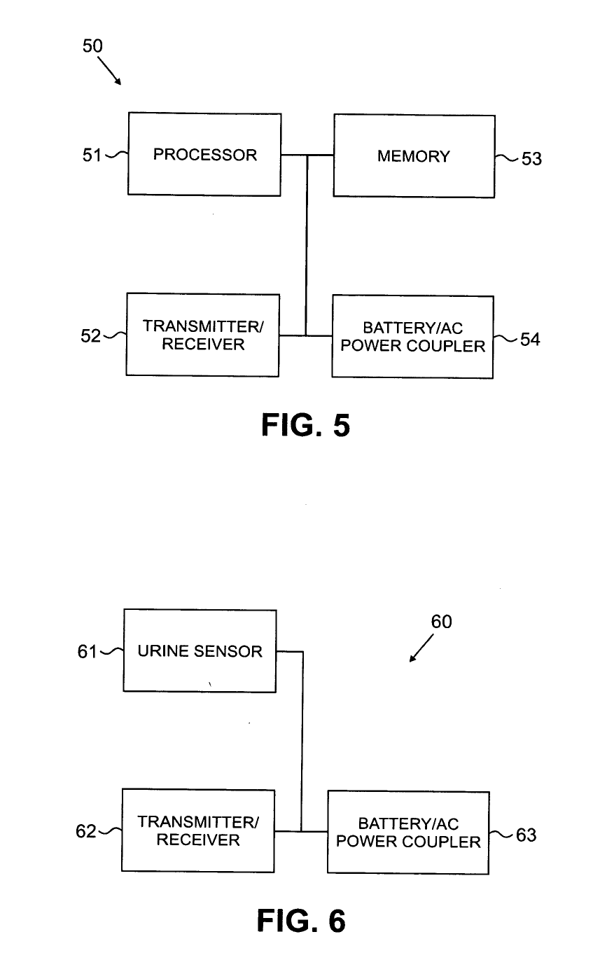

An exemplary embodiment of a central recorder unit 50 is shown in FIG. 5. The recorder unit 50 includes a memory 53 for storing all received data along with time stamps, a processor 51 for controlling the various sensors and memory logging, a transmitter/receiver for communicating with the various sensors, and a battery/AC power coupler 54 to power the recorder unit 50. The battery can be rechargeable by connecting the battery 54 to an AC power source. The processor can be programmed by downloading a program into the memory 53. Transmitter/receiver 52 can be an RF transmitter at a frequency selected to communicate easily with the various sensors, such as 800 MHz or so.

### Leak Sensor

Additionally, a leak sensor 14 could be placed near the outlet of the urethra, and be wired or otherwise coupled to the central unit 12 to transmit the occurrence of an incontinence episode to the recorder unit 12. The leak point pressure that resulted in the episode could be derived from the information derived from all of the sensors 11, 13, 14. Alternatively, the leak sensor could be wirelessly coupled to the central unit 12 to transmit any data collected.

Several commercial urine sensors exist. For example, QuickMedical sells a Bedwetting Alarm kit that employs a urine sensor that activates an alarm. The urine sensor in this system could be employed to activate a transmitter to transmit the presence of urine to the central unit 12 to obtain correlation of data from the other sensors to enable a complete “picture” of the forces acting before, during and after an incontinence episode.

An exemplary embodiment of a leak sensor is shown in FIG. 6. A urine sensor 61 is coupled to a battery/AC power coupler 63 and to a transmitter, which transmits upon urine sensor sensing the presence of urine. Since the urine sensor is merely a positive indication, the transmission can be relatively simple so that a burst transmission indicates presence of urine at the urine sensor. If desired, the battery can be a simple rechargeable battery given that this sensor 60 is disposed outside of the patient's body.

### Flow Rate Sensor

A standard flow rate sensor 15 can be disposed in the patient's toilet and wirelessly coupled to the recorder unit so that when the patient urinates, flow data is transmitted in real-time to the recorder unit on the patient's waist. Flow data obtained when another is urinating can be prevented from being received by the recorder unit, for example, by limiting the range of the transmission to ensure that only the patient's flow rate data is received by the recorder unit. Moreover, inadvertent reception can be identified by lack of volume changes in the bladder at the instance of the reception of flow rate data, thereby preventing corruption of the data, even when the recorder unit records data measurements from the wrong patient.

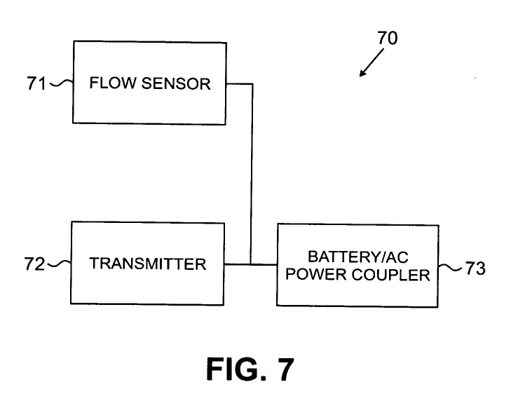

An exemplary embodiment of a flow rate sensor is shown in FIG. 7. A flow rate sensor 71 is coupled to a battery/AC power coupler 73 and to a transmitter 72, which transmits upon a positive flow rate being detected by the flow rate sensor. If desired, the battery can be a rechargeable battery given that this sensor 70 is disposed outside of the patient's body.

### Operation

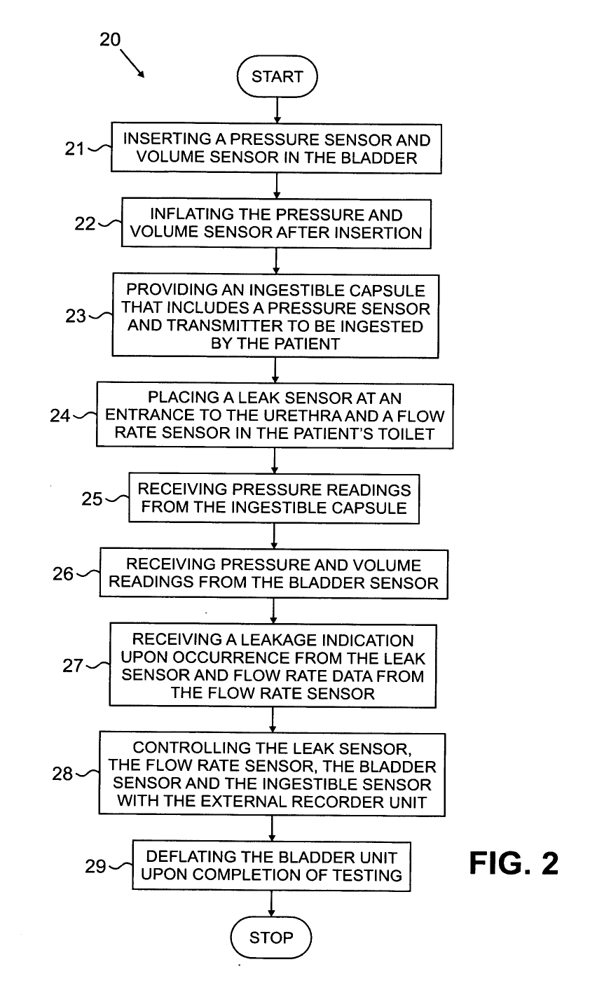

Turning to FIG. 2, shown therein is an exemplary embodiment of a method for obtaining urological data from a patient while the patient is undergoing his or her normal routine according to one aspect of the present invention.

At step 21, a pressure and volume sensor is inserted manually into the bladder by an urologist or other physician or trained practitioner. The bladder unit measures volume and pressure within the bladder and transmits this data to a recorder unit, for example, worn around the waist of the patient.

At step 22, after insertion into the bladder, the bladder unit is inflated to maintain the unit inside the bladder while the testing is occurring. By inflating the bladder unit to a size larger than the hole in the bladder, normal urinary activities will not discharge the bladder unit.

At step 23, an ingestible capsule is provided to the patient, which capsule includes a pressure sensor and a transmitter, along with the necessary battery and controls to enable remote activation and interrogation of the sensor while the sensor passes through the gastrointestinal tract. One or more capsules can be ingested at predetermined times to enable a continuous range of pressure data readings. Other possible data can be obtained as desired, including temperature, acidity (PH), etc.

At step 24, a leak sensor is placed at the entrance to the urethra to detect the presence of urine. This sensor transmits an indication of the presence of urine to the recorder unit so that pressure and volume readings from the other sensors can be correlated with urinary activities. In addition, a urine flow sensor is mounted to the patient's toilet to enable flow rate measurements to be obtained while the patient is urinating into the sensor. This sensor transmits the flow rate data to the recorder unit, which is in close proximity to the flow sensor.

At step 25, pressure readings are received from the ingestible capsule. These readings can be obtained from RF transmissions from the ingestible capsule.

At step 26, pressure and volume readings are received from the bladder unit. These readings can be obtained from RF transmissions from the bladder unit.

At step 27, upon occurrence, leakage detection signals are received from the leakage sensor. Flow rate data is also received during urination from the flow rate sensor. These readings can be obtained from RF transmissions from the leakage sensor and the flow rate sensor.

At step 28, the recorder unit controls the various sensors. This can be accomplished by transmission of control signals via RF to each of the various sensors. These control signals can be interrogation signals to indicate that the sensor should upload all collected data, activation signals to turn on the various sensors to initiate data collection, and/or signals to cause inflation or deflation of the bladder unit.

At step 29, the bladder unit is deflated upon completion of testing so that the bladder unit can be passed out of the bladder in the normal course of urination.

### SUMMARY

Although various embodiments are specifically illustrated and described herein, it will be appreciated that modifications and variations of the invention are covered by the above teachings and are within the purview of the appended claims without departing from the spirit and intended scope of the invention. For example, the above discussion relates to transmission of data from the various sensors to the recorder unit in a wireless RF manner, however, other transmission methods are possible. In addition, certain techniques are discussed for obtaining volume and pressure measurements, however other techniques are also possible. Furthermore, these examples should not be interpreted to limit the modifications and variations of the invention covered by the claims but are merely illustrative of possible variations.

1. An apparatus for obtaining data from a patient comprising: (a) an ingestible capsule to sense pressure and to transmit pressure measurements from a gastrointestinal tract of the patient; (b) a bladder unit to sense pressure and to transmit bladder volume data from a bladder of the patient; (c) a leak sensor to be disposed at an outlet of the urethra to sense a presence of urine and to transmit an indication of such presence; and (d) a body worn recorder unit to receive pressure measurements from the ingestible capsule, pressure and volume measurements from the bladder unit, and urine presence indications from the leak sensor and to record the measurements.

2. The apparatus according to claim 1, wherein the bladder unit further comprises: (a) an oil-filled reservoir to float the bladder unit against a wall of the bladder; (b) a ultrasonic sensor to transmit and receive an ultrasonic signal; (c) a processor to measure a time needed to receive a reflection of the transmitted ultrasonic signal, thereby measuring a distance to an opposite wall of the bladder; and (d) a transmitter to transmit the time to the recorder unit.

3. The apparatus according to claim 1, wherein the ingestible capsule further comprises: (a) a pressure sensor to measure pressure; and (b) a transmitter to transmit pressure data to the recorder unit.

4. The apparatus according to claim 1, wherein the leak sensor further comprises: (a) a leakage sensor to detect a presence of urine; and (b) a transmit to transmit urine presence signals to the recorder unit.

5. The apparatus according to claim 1, wherein the body worn recorder unit further comprises a memory to store received data from each of the bladder unit, the ingestible capsule, and the leakage sensor.

6. The apparatus according to claim 1, further comprising a flow rate sensor disposed in a toilet of the patient and transmitting flow rate data to the recorder unit during patient urination.

7. The apparatus according to claim 6, wherein the flow rate sensor further comprises a transmitter transmitting flow rate data to the recorder unit.

8. The apparatus according to claim 1, wherein the bladder unit further comprises: (a) an inflatable housing that is remotely inflatable after insertion of the bladder unit into the bladder; (b) a gas source to emit a gas upon remote activation to inflate the inflatable housing; and (c) a degradable suture sewn into the inflatable housing to maintain an airtight seal around the housing, the degradable suture deteriorating after a predetermined time inside the bladder and causing deflation of the inflatable housing upon the deterioration.

9. The apparatus according to claim 1, wherein the gas source for the bladder unit further comprises: (a) a first compartment; (b) a liquid stored in the first compartment; (c) a second compartment; (d) a plurality of gas-releasing granules stored in the second compartment; (e) a heat breakable seal separating the first compartment from the second compartment; (f) an electric current path disposed along the seal; and (g) a remotely activatable power source coupled to the electric current path to cause an electric current to pass through the electric current path disposed along the seal and to heat the seal thereby breaking the seal and merging the liquid with the granules.

10. A method for obtaining urodynamic data from a patient comprising: (a) coupling pressure information from an ingestible capsule disposed in a gastrointestinal tract of the patient to a body worn recorder unit disposed on the patient's body; (b) coupling pressure and volume information from a unit disposed in a bladder of the patient to the body worn recorder unit; and (c) coupling leak sense information from a leak sensor disposed at an entrance to the patient's urethra to the body worn recorder unit.

11. The method according to claim 10, further comprising inflating remotely the bladder unit after inserting the bladder unit into the bladder.

12. The method according to claim 10, further comprising storing pressure and volume data received from the ingestible capsule and the bladder unit in the recorder unit.

13. A method for obtaining urological data from a patient comprising: (a) inserting a pressure sensor and volume sensor in the bladder; and (b) inflating the pressure and volume sensor after insertion.

14. The method according to claim 13, further comprising providing an ingestible capsule that includes a pressure sensor and transmitter to be ingested by the patient.

15. The method according to claim 13, further comprising placing a leak sensor at an entrance to the urethra.

16. The method according to claim 13, further comprising placing a flow rate sensor in the patient's toilet.

17. The method according to claim 14, further comprising receiving pressure readings from the ingestible capsule.

18. The method according to claim 13, further comprising receiving pressure and volume readings from the bladder sensor.

19. The method according to claim 15, further comprising receiving a leakage indication upon occurrence from the leak sensor.

20. The method according to claim 16, further comprising receiving flow rate data from the flow rate sensor.

21. The method according to claim 13, further comprising: (a) providing an ingestible capsule that includes a pressure sensor and transmitter to be ingested by the patient; (b) placing a leak sensor at an entrance to the urethra; (c) placing a flow rate sensor in the patient's toilet; and (d) controlling the leak sensor, the flow rate sensor, the bladder sensor and the ingestible sensor with an external recorder unit.

22. The method according to claim 13, further comprising deflating the bladder unit upon completion of testing.
